(19) United States
(12) Patent Application Publication
(10) Pub. No.: US 2013/0315973 A1
(43) Pub. Date: Nov. 28, 2013

(54) BIOCOMPATIBLE DEVICES COATED WITH A TRIBONECTIN AND METHODS FOR THEIR PRODUCTION

(71) Applicant: Gregory D. Jay, Norfolk, MA (US)

(72) Inventor: Gregory D. Jay, Norfolk, MA (US)

(21) Appl. No.: 13/472,938

(22) Filed: May 16, 2012

Related U.S. Application Data
(63) Continuation of application No. 12/210,000, filed on Sep. 12, 2008, now abandoned.
(60) Provisional application No. 60/993,553, filed on Sep. 12, 2007.

(51) Int. Cl. A61L 31/10; A61L 31/16

(52) U.S. Cl. CPC A61L 31/10; A61L 31/16
USPC 424/423; 514/1.1; 514/19.2; 424/278.1

(57) ABSTRACT

The present invention features biocompatible devices having a surface thereof coated with a composition that includes a tribonectin, and methods of making the devices. The tribonectin may, e.g., reduce microbial growth on or attachment to the surface of the biocompatible device.

### CROSS REFERENCE TO RELATED APPLICATIONS

This application claims priority from U.S. provisional application 60/993,553, filed Sep. 12, 2007, which is incorporated herein by reference.

### BACKGROUND OF THE INVENTION

The implantation of biocompatible devices has become routine in most areas of critical care practice, anesthesia, and management of patients with a variety of illnesses. These biocompatible devices are frequently conduits for infection. Central line sepsis of an implanted catheter is one of the most frequently acquired complications and a potentially life-threatening event for a patient. Therefore, developing strategies for the prevention of microbial growth on the surface of a biocompatible device is necessary.

Biocompatible devices that are coated or impregnated with antimicrobial agents may decrease the risk of, e.g., bacterial or fungal infections. Chlorhexidine, silver sulfadiazine, ionic metals (e.g., platinum and silver), and other antibiotics (e.g., rifampin, minocycline, and vancomycin) have been used to coat the surfaces of biocompatible devices. However, these antimicrobials often have a short half-life. For example, the half-life of antimicrobial activity of chlorhexidine/silver sulfadiazine on the surface of a device is three days in vitro when tested against Staphylococci epidermis, while the half-life of antimicrobial activity against Staphylococci epidermidis is twenty-five days in vitro for a device coated with minocycline or rifampin. Most biocompatible devices implanted in a patient remain for significantly longer than one week. Thus, there exists a need in the art for improved biocompatible devices that are resistant to microbial growth and methods for making such a device.

### SUMMARY OF THE INVENTION

The devices and methods of the invention are directed to coating the surface of a biocompatible device with a tribonectin. In one embodiment, a biocompatible device includes a surface layer coating containing a tribonectin, which is adapted for use within the body of a mammal. Preferably, the biocompatible device is a non-diarthrodial device. The device of may be used for the reduction of microbial growth on the surface of the biocompatible device for use within a mammal in need of the device. Preferably, the tribonectin is present in an amount sufficient to reduce microbial growth on the surface of the device when used within a mammal. The concentration of tribonectin in the coating may be, e.g., between 0.1 μg/ml to 1.0 mg/ml. Preferably, the concentration of tribonectin is 0.2 mg/ml. The tribonectin may be, e.g., lubricin or a biologically active fragment thereof. Preferably, the device is sterile.

The coating of the biocompatible device may further include a biologically active agent. The biologically active agent may be, e.g., an anti-inflammatory agent, antimicrobial agent, antifungal agent, antiviral agent, antiproliferative agent, analgesic, anesthetic, immunomodulator, or a lubricant. The anti-inflammatory agent may be, e.g., ibuprofen, tacrolimus, rofecoxib, celecoxib, flubiprofen, diclofenac, or ketarolac. The antimicrobial agent may be, e.g., penicillin, ampicillin, methicillin, oxacillin, amoxicillin, cefadroxil, ceforanid, cefotaxime, ceftriaxone, doxycycline, minocycline, tetracycline, amikacin, gentamycin, kanamycin, neomycin, streptomycin, tobramycin, azithromycin, clarithromycin, erythromycin, ciprofloxacin, lomefloxacin, moxifloxacin, norfloxacin, chloramphenicol, clindamycin, cycloserine, isoniazid, rifampin, or vancomycin. The antiviral agent may be, e.g., ribavirin, 9-2-hydroxy-ethoxy methylguanine, adamantanamine, 5-iodo-2′-deoxyuridine, trifluorothymidine, interferon, adenine arabinoside, acyclovir, penciclovir, valacyclovir, or ganciclovir. The antiproliferative agent may be, e.g., asparaginase, bleomycin, busulfan carmustine (BCNU), chlorambucil, cladribine (2-CdA), CPT11, cyclophosphamide, cytarabine (Ara-C), dacarbazine, daunorubicin, dexamethasone, doxorubicin (adriamycin), etoposide, fludarabine, 5-fluorouracil (5FU), hydroxyurea, idarubicin, ifosfamide, interferon-α (native or recombinant), levamisole, lomustine (CCNU), mechlorethamine (nitrogen mustard), melphalan, mercaptopurine, methotrexate, mitomycin, mitoxantrone, paclitaxel, pentostatin, prednisone, procarbazine, tamoxifen, taxol-related compounds, 6-thioguanine, topotecan, vinblastine, or vincristine. The antifungal agent may be, e.g., amphotericin B, butylparaben, clindamycin, econaxole, fluconazole, flucytosine, griseofulvin, nystatin, or ketoconazole. The analgesic may be, e.g., morphine, codeine, hydrocodone, oxycodone, acetaminophen, aspirin, codeine, naproxen, or ibuprofen. The anesthetic may be, e.g., procaine, lidocaine, tetracaine, dibucaine, benzocaine, p-buthylaminobenzoic acid 2-(diethylamino) ethyl ester HCl, mepivacaine, piperocaine, or dyclonine. The lubricant may be, e.g., hyaluronic acid, a proteoglycan, chondroitin sulfate, a cellulose derivative, hydroxypropylmethyl cellulose, carboxymethyl cellulose, methyl cellulose, hydroxyethyl cellulose, collagen, a viscosifier, polyvinyl alcohol, polyvinylpyrrolidone, or a carboxyvinyl polymer. Preferably, the lubricant is hyaluronic acid. The hyaluronic acid may be present in the coating at a concentration of between 0.1 mg/ml to 50.0 mg/ml. The immunomodulator may be, e.g., ascomycin, cyclosporine, everolimus, pimecrolimus, rapamycin, tacrolimus, beclomethasone, budesonide, dexamethasone, fluorometholone, fluticasone, hydrocortisone, loteprednol etabonate, medrysone, rimexolone, or triamcinolone.

The biocompatible device may be, e.g., a catheter, stent, intraocular lens, dialysis graft, pacemaker lead, implantable defibrillator, suture, suture anchor, staple, clamp, screw, plate, shunt, bone pin, vertebral disk, hemostatic barrier, tissue adhesive or sealant, tissue scaffold, bone substitute, anastomosis device, intraluminal device, angioplasty device, drug-delivery device, non-diarthrodial prosthetic implant, vascular implant, or vascular support. Preferably, the biocompatible device is a non-diarthrodial device.

In another embodiment, the invention features a method of making a biocompatible device adapted for use within the body of a mammal including the steps of coating a surface layer of the biocompatible device with a tribonectin. The method may be used to reduce microbial growth on the surface of the device for use within a mammal in need of the device. Preferably, the method includes a tribonectin present in an amount sufficient to reduce microbial growth on the surface of the device when used within a mammal. The concentration of the tribonectin in the coating may be, e.g., between 0.1 μg/ml to 1.0 mg/ml. The tribonectin may be, e.g., lubricin or a biologically active fragment thereof. Preferably, the device is sterile.

The coating of the biocompatible device of the methods described herein may further include a biologically active agent. The biologically active agent may be, e.g., an anti-inflammatory agent, antimicrobial agent, antifungal agent, antiviral agent, antiproliferative agent, analgesic, anesthetic, immunomodulator, or a lubricant. The anti-inflammatory agent may be, e.g., ibuprofen, tacrolimus, rofecoxib, celecoxib, flubiprofen, diclofenac, or ketarolac. The antimicrobial agent may be, e.g., penicillin, ampicillin, methicillin, oxacillin, amoxicillin, cefadroxil, ceforanid, cefotaxime, ceftriaxone, doxycycline, minocycline, tetracycline, amikacin, gentamycin, kanamycin, neomycin, streptomycin, tobramycin, azithromycin, clarithromycin, erythromycin, ciprofloxacin, lomefloxacin, moxifloxacin, norfloxacin, chloramphenicol, clindamycin, cycloserine, isoniazid, rifampin, or vancomycin. The antiviral agent may be, e.g., ribavirin, 9-2-hydroxy-ethoxy methylguanine, adamantanamine, 5-iodo-2′-deoxyuridine, trifluorothymidine, interferon, adenine arabinoside, acyclovir, penciclovir, valacyclovir, or ganciclovir. The antiproliferative agent may be, e.g., asparaginase, bleomycin, busulfan carmustine (BCNU), chlorambucil, cladribine (2-CdA), CPT11, cyclophosphamide, cytarabine (Ara-C), dacarbazine, daunorubicin, dexamethasone, doxorubicin (adriamycin), etoposide, fludarabine, 5-fluorouracil (5FU), hydroxyurea, idarubicin, Ifosfamide, interferon-α (native or recombinant), levamisole, lomustine (CCNU), mechlorethamine (nitrogen mustard), melphalan, mercaptopurine, methotrexate, mitomycin, mitoxantrone, paclitaxel, pentostatin, prednisone, procarbazine, tamoxifen, taxol-related compounds, 6-thioguanine, topotecan, vinblastine, or vincristine. The antifungal agent may be, e.g., amphotericin B, butylparaben, clindamycin, econaxole, fluconazole, flucytosine, griseofulvin, nystatin, or ketoconazole. The analgesic may be, e.g., morphine, codeine, hydrocodone, oxycodone, acetaminophen, aspirin, codeine, naproxen, or ibuprofen. The anesthetic may be, e.g., procaine, lidocaine, tetracaine, dibucaine, benzocaine, p-buthylaminobenzoic acid 2-(diethylamino) ethyl ester HCl, mepivacaine, piperocaine, or dyclonine. The lubricant may be, e.g., hyaluronic acid, a proteoglycan, chondroitin sulfate, a cellulose derivative, hydroxypropylmethyl cellulose, carboxymethyl cellulose, methyl cellulose, hydroxyethyl cellulose, collagen, a viscosifier, polyvinyl alcohol, polyvinylpyrrolidone, or a carboxyvinyl polymer. Preferably, the lubricant is hyaluronic acid. The hyaluronic acid may be present in the coating at a concentration of between 0.1 mg/ml to 50.0 mg/ml. The immunomodulator may be, e.g., ascomycin, cyclosporine, everolimus, pimecrolimus, rapamycin, tacrolimus, beclomethasone, budesonide, dexamethasone, fluorometholone, fluticasone, hydrocortisone, loteprednol etabonate, medrysone, rimexolone, or triamcinolone.

The biocompatible device described in the methods may be, e.g., a catheter, stent, intraocular lens, dialysis graft, pacemaker lead, implantable defibrillator, suture, suture anchor, staple, clamp, screw, plate, shunt, bone pin, vertebral disk, hemostatic barrier, tissue adhesive or sealant, tissue scaffold, bone substitute, anastomosis device, intraluminal device, angioplasty device, drug-delivery device, non-diarthrodial prosthetic implant, vascular implant, or vascular support. Preferably, the biocompatible device is a non-diarthrodial device.

By “an amount sufficient” is meant the amount of an agent (e.g., a tribonectin or a biologically active agent) required to improve, inhibit, or ameliorate a condition (e.g., infection with one or more microbial agents) in a mammal (e.g., a human patient) in a clinically relevant manner. For example, a sufficient amount of tribonectin, is an amount capable of reducing microbial growth on, or attachment of microbes to, the surface of a biocompatible device by at least 10%, preferably at least about 20%, 30°/u, 40%, more preferably by at least 50%, 60%, 70%, and most preferably by at least 80%, 90%, 95%, or more (e.g., 100%).

By “biocompatible device” is meant that the device is substantially non-toxic to a mammalian body and does not significantly induce inflammation or other adverse responses. Biocompatible devices include, but are not limited to, e.g., a catheter, stent, intraocular lens, dialysis graft, pacemaker lead, implantable defibrillator, suture, suture anchor, staple, clamp, screw, plate, shunt, bone pin, vertebral disk, hemostatic barrier, tissue adhesive or sealant, tissue scaffold, bone substitute, anastomosis device, intraluminal device, angioplasty device, drug-delivery device, prosthetic implant, vascular implant, or vascular support. Preferably, the biocompatible device is a non-diarthrodial device.

By “biologically active agent” is meant any agent that produces a preventative, healing, curative, stabilizing, ameliorative or other beneficial therapeutic effect.

By “microbe” is meant a bacterium or a fungus. By “microbial” is meant of or relating to bacteria or fungi. Exemplary bacteria include, e.g., staphylococci (e.g., Staphylococcus epidermidis or Staphylococcus aureus), Enterococcus faecalis, Pseudomonas aeruginosa, Escherichia coli, Klebsiella pneumoniae, and other gram-positive and gram-negative bacteria. A fungus may be, e.g., Candida albicans, Candida glabrata, Aspergillus flavus, Aspergillus fumigatus, Aspergillus glaucus, Aspergillus nidulans, Aspergillus niger, Aspergillus terreus, Blastomyces dermatitidis, Coccidioides immitis, Coccidioides posadasii, Cryptococcus neoformans, Histoplasma capsulatum, Paracoccidioides brasiliensis, Sporothrix schenckii, Absidia corymbifera, Rhizomucor pusillus, and Rhizopus arrhizus.

By “non-diarthrodial device” is meant any device that is not adapted for use as a diarthrodial joint (e.g., a freely moveable joint), or a device that is not adapted for use within a diarthrodial joint.

By “patient” is meant any mammal (e.g., a human). A patient who is being treated using a biocompatible device described herein may be one who has been diagnosed by a medical practitioner as being in need of such a device. Diagnosis may be performed by any suitable means. One skilled in the art will understand that patients described herein may have been subjected to standard tests or may have been identified, without examination, as one at high risk due to the presence of one or more risk factors, such as age or a family history of a disease.

The terms “polypeptide” and “peptide” are used interchangeably and refer to any chain of more than two natural or unnatural amino acids, regardless of post-translational modification (e.g., glycosylation or phosphorylation), constituting all or part of a naturally-occurring or non-naturally occurring polypeptide or peptide.

By “substantially identical” is meant a polypeptide or nucleic acid exhibiting at least 60%, 65%, 70%, 75%, 80%, 85%, 90%, 95%, 98%, 99%, or even 100% identity to a reference amino acid or nucleic acid sequence over at least 20, 25, 30, 35, 40, 45, 50, 55, 60, 65, or 70 contiguous residues or bases.

By “tribonectin” is meant a mucinous glycoprotein that is substantially identical to proteoglycan 4 (PRG4), articular cartilage superficial zone protein (SZP), megakaryocyte stimulating factor precursor, or lubricin.

### BRIEF DESCRIPTION OF THE DRAWINGS

FIG. 1 is the amino acid sequence of megakaryocyte stimulating factor precursor.

FIG. 2 is the cDNA sequence of megakaryocyte stimulating factor precursor.

### DETAILED DESCRIPTION

The present invention features devices and methods for coating the surface of a biocompatible device with tribonectin. The tribonectin promotes a reduction in microbial attachment to or growth on the surface of the biocompatible device.

### Tribonectins

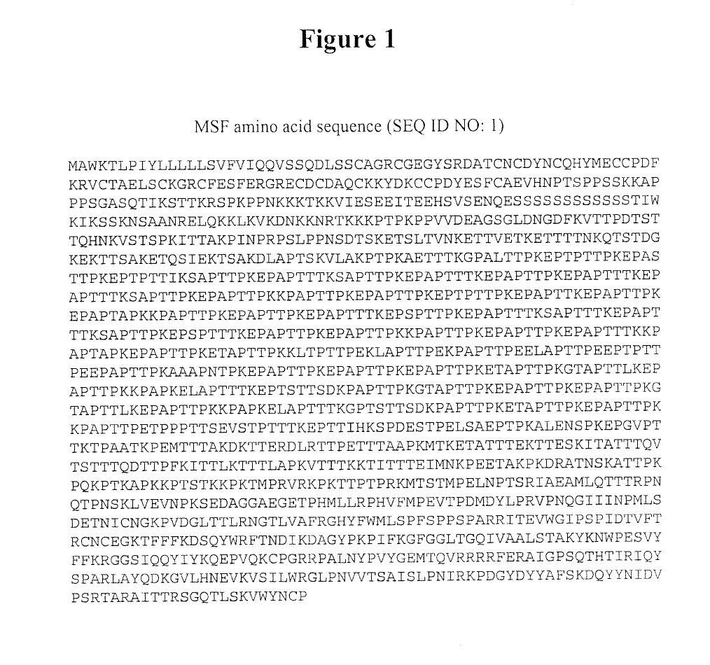

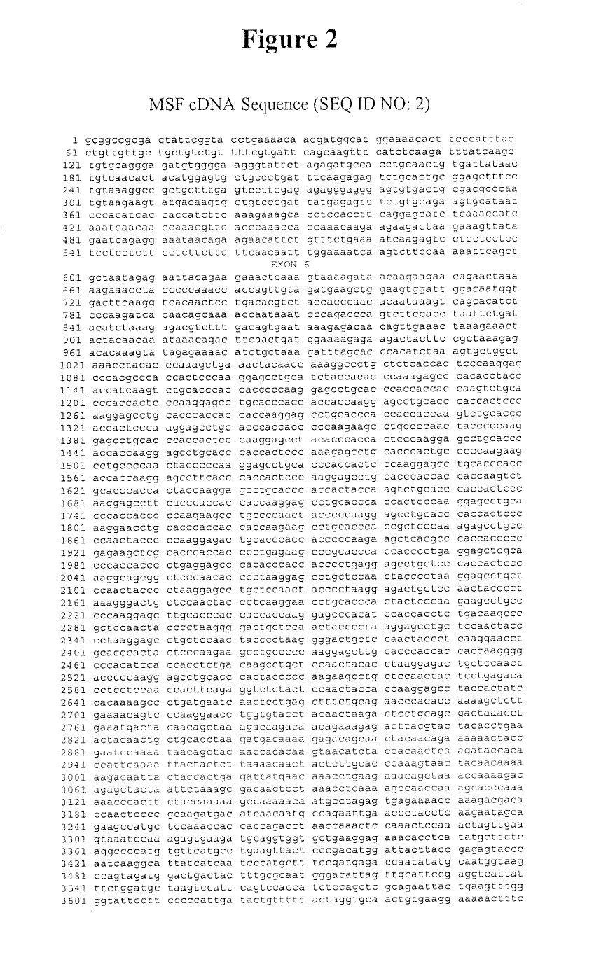

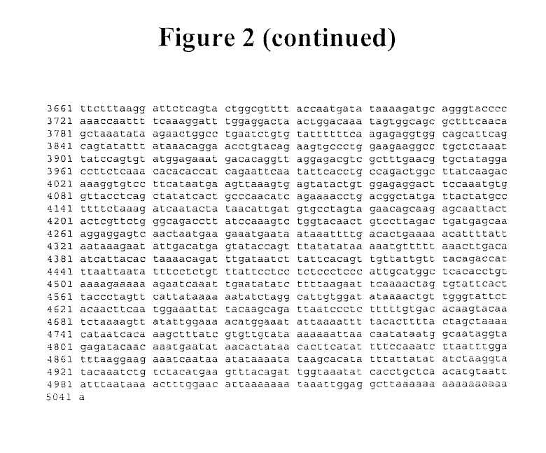

A tribonectin lubricin) is a lubricating polypeptide that contains at least one repeat of an amino acid sequence that is at least 50% identical to KEPAPTT (SEQ ID NO: 3). Tribonectins are substantially identical in sequence to all or a portion of the amino acid or nucleic acid sequences of proteoglycan 4 (PRG4), articular cartilage superficial zone protein (SZP), megakaryocyte stimulating factor precursor, and lubricin. The amino acid and cDNA sequences of megakaryocyte stimulating factor precursor are described in FIGS. 1 and 2, respectively. A tribonectin is glycosylated by at least one O-linked oligosaccharide lubricating moiety, e.g., an N-acetylgalactosamine and galactose in the form β(1-3)Gal-GalNAC. The β(1-3)Gal-GalNAc may be capped with NeuAc. The term “glycosylated” means that a carbohydrate moiety is present at one or more sites on the polypeptide molecule. For example, at least 10%, preferably at least 20%, more preferably at least 30%, and most preferably at least 40% of the tribonectin is glycosylated. Fifty percent or more of the tribonectin may be glycosylated. The percentage of glycosylation is determined by weight.

One characteristic of a tribonectin is the ability to reduce the coefficient of friction (μ) between bearing surfaces. For example, reduction of friction is measured in vitro by detecting a reduction in friction in a friction apparatus using latex-glass bearings. Reduction of friction is also measured in vivo, e.g., by measuring reduction of pain in a patient.

In addition to serving as a lubricating composition, a tribonectin, as described in the invention herein, can be coated onto the surface (e.g., an interior or exterior surface) of a biocompatible device to prevent the growth of microbes on the device, or their attachment to the device (see, e.g., Example 1). For example, tribonectins may have antimicrobial activity, and thus, may reduce the attachment or growth of bacteria (e.g., Staphylococcus epidermidis, Staphylococcus aureus, Enterococcus faecalis, Pseudomonas aeruginosa, Escherichia coli, Klebsiella pneumoniae, and other gram-positive and gram-negative bacteria) on the surface of a biocompatible device when applied to a surface of the device as a coating. Tribonectins applied to the surface of a biocompatible device may also reduce the attachment or growth of fungi (e.g., Candida albicans, Candida glabrata, Aspergillus flavus, Aspergillus fumigatus, Aspergillus glaucus, Aspergillus nidulans, Aspergillus niger, Aspergillus terreus, Blastomyces dermatitidis, Coccidioides immitis, Coccidioides posadasii, Cryptococcus neoformans, Histoplasma capsulatum, Paracoccidioides brasiliensis, Sporothrix schenckii, Absidia corymbifera, Rhizomucor pusillus, and Rhizopus arrhizus) on the surface of the biocompatible device.

The production and purification of tribonectins for use in the invention described herein are described in U.S. Pat. No. 7,001,881, hereby incorporated by reference. Briefly, the tribonectin may be a recombinant protein. Expression systems that may be used for purposes of the invention include, e.g., microorganisms such as bacteria (e.g., E. coli and B. subtilis) transformed with recombinant bacteriophage DNA, plasmid DNA, or cosmid DNA expression vectors containing a nucleic acid molecule encoding the tribonectin. For production of glycosylated polypeptides, eukaryotic expression systems may be used. Yeast (e.g., Saccharomyces and Pichia) transformed with recombinant yeast expression vectors containing the recombinant nucleic acid encoding a tribonectin polypeptide may be used. Insect cell systems infected with recombinant virus expression vectors (e.g., baculovirus) containing the nucleic acid molecules encoding a tribonectin and mammalian cell systems (e.g., COS, CEO, BEK, 293, VERO, HeLa, MDCK, W138, and NIH 3T3 cells) harboring recombinant expression constructs containing promoters derived from the genome of mammalian cells (e.g., the metallothionein promoter) or from mammalian viruses (e.g., the adenovirus late promoter and the vaccinia virus 7.5 K promoter) may also be useful. Tribonectin analogs, mimetics, and isoforms, for use in the invention described herein, may also be produced and purified using these methods.

Examples of tribonectins, for use in the devices and methods of the invention, are described in, e.g., U.S. Pat. Nos. 6,743,774, 6,960,562, and 7,001,881, as well as U.S. Patent Publication Nos. 2004/0072741, 2004/0229804, and 2007/0111327, hereby incorporated by reference.

### Biocompatible Devices

Any biocompatible device may be used in the invention described herein. For example, devices suitable for contact with, e.g., bodily fluids, may be used. The duration of contact may be short-term (e.g., surgical instruments) or long-term (e.g. implants). Biocompatible devices that can be coated with a tribonectin, according to the invention, include, but are not limited to, e.g., a catheter, stent, intraocular lens, dialysis graft, pacemaker lead, implantable defibrillator, suture, suture anchor, staple, clamp, screw, plate, shunt, guide wire, bone pin, tubing, vertebral disk, hemostatic barrier, tissue adhesive or sealant, tissue scaffold, bone substitute, anastomosis device, intraluminal device, angioplasty device, drug-delivery device, prosthetic implant, vascular implant, or vascular support. Preferably, the device is a non-diarthrodial device.

The biocompatible device may be an implanted device, a percutaneous device, or a cutaneous device. Implanted devices, which are fully implanted into a patient, include, e.g., prosthetic implants (e.g., non-diarthrodial prosthetic implants), electrical leads (e.g., pacemaker leads), implantable defibrillators, artificial heart valves, heart valve stents, coronary stents, vascular and structural stents, vascular or cardiovascular shunts, biological conduits, pledges, sutures, annuloplasty rings, staples, dermal grafts for wound healing, orthopedic spinal implants, orthopedic pins, intrauterine devices, urinary stents, intraocular lenses, and drug-delivery devices. Percutaneous devices penetrate the skin, thereby extending from outside the body into the body. Percutaneous devices include, e.g., catheters, cannulas, drainage tubes (e.g., chest tubes), surgical instruments (e.g., forceps, retractors, or needles), and catheter cuffs. Cutaneous devices, used superficially, include, e.g., burn dressings, wound dressings, patches (e.g., hernia patches), and dental hardware (e.g., bridge supports and bracing components).

Coating of the Biocompatible Device

The biocompatible device of the invention may be, e.g., coated with a tribonectin and, optionally, a biologically active agent. Methods for coating biocompatible devices are described in, e.g., U.S. Pat. Nos. 5,702,456, 5,709,020, 5,824,048, 6,153,252, 6,258,121, and 7,056,550, hereby incorporated by reference. To coat the biocompatible device, the device may be contacted with, e.g., a solution containing a solvent, a tribonectin, and, optionally, a biologically active agent, by dipping, spraying, soaking, or otherwise applying the solution to the surface of the biocompatible device. The biocompatible device may be contacted with the solution for a short period of time (e.g., 5 minutes, 10 minutes, 20 minutes, or 30 minutes) or, alternatively, may be incubated in the solution for several hours (e.g., one hour, two hours, three hours, or longer). The solvent may be, e.g., a standard biological buffer (e.g., a low ionic strength aqueous buffer at near-neutral pH, such as Tris-buffered saline (TBS), or physiologic saline) or a biocompatible organic solvent. The contact or incubation of the biocompatible device to or in the solution, respectively, may be performed at a temperature at which the biocompatible device and the solution are not degraded or denatured. The biocompatible device may have one layer of a tribonectin coating or may have multiple layers of a tribonectin coating. Alternatively, the biocompatible device may have one or more layers of a tribonectin coating in addition to one or more layers of a different coating (e.g., a coating containing a biologically active agent). After the device has been coated, the device may be dried (e.g., at an ambient temperature or by heating). The device may also be sterilized prior to its use within the body of a mammal.

Although polymeric carriers are not required for the attachment of a tribonectin to the surface of the biocompatible device, polymeric carriers may be used for this purpose. Polymeric carriers may include, e.g., polyurethanes, polyvinyls, polycarboxylic acids (e.g., polyacrylic acid, polymethacrylic acid, and polymaleic acid) acrylic or methacrylic copolymers (e.g., poly(ethylene-co-acrylic acid), cellulose-derived polymers (e.g., nitrocellulose, cellulose acetate butyrate, cellulose acetate propionate), polyamines, polysulfonates, polycarbonates, and acrylate and methacrylate copolymers (e.g., poly(ethylene-co-vinyl acetate)), as well as blends thereof. Linear copolymers, cross-linked copolymers, graft polymers, and block copolymers may also be used in a polymeric coating. The tribonectin or biologically active agent may also be incorporated into a calcium phosphate or hydroxyapatite coating applied onto the biocompatible device. The tribonectin or additional biologically active agent may also be incorporated into the biocompatible device during the production or shaping of the device, provided that the tribonectin or additional biologically active agent is stable and remains functional at the conditions (e.g., temperature and pressure) required during such production or shaping.

As biocompatible devices are made in a variety of configurations and sizes, the exact concentration or amount of e.g., a tribonectin and, optionally, a biologically active agent, present on the surface of the device, will vary with the size of the device, surface area, design, portions of the biocompatible device being coated, the length of time during which the biocompatible device is intended to remain in the mammal, and the rate at which the therapeutic agent is released from the device. The amount of a tribonectin or biologically active agent may be calculated as a function of concentration per unit area of the portion of the device being coated, total amount coated onto the device may then be measured, and the appropriate surface concentrations of the tribonectin and, optionally, the biologically active agent may be determined. The concentration of the tribonectin used to coat the surface of the biocompatible device may be, e.g., between 0.1 μg/ml and 1.0 mg/ml (e.g., 0.2 mg/ml).

The ability of a tribonectin to reduce microbial growth on the surface of a biocompatible device may be measured by one of several methods known in the art (see, e.g., U.S. Pat. Nos. 5,366,505, 6,054,504, and 6,514,517, hereby incorporated by reference). Once the device is coated or impregnated with a tribonectin and, optionally, a biologically active agent, the device may be exposed to a microbial source over a specified period of time, after which the device is washed and the growth of the microbe on the device is measured. Such measurements may include colony counts of the microbe or other means of quantifying microbial growth, such as chemiluminescent or bioluminescent assays, which monitor a particular metabolite of the microbe as a means of quantifying microbial growth. Alternatively, microbial growth may be monitored, through radiolabelling techniques. One method for analyzing the effectiveness of tribonectin in preventing microbial growth on the surface of a biocompatible device is described in Example 1.

The biocompatible device of the invention described herein may be adapted to be used for a short period of time (e.g., less than thirty days) or may be adapted for use as a long-term implant (e.g., from a period of more than thirty days to one year or longer).

### Biologically Active Agents

If desired, the surface layer of the biocompatible device may include, in addition to the tribonectin, a biologically active agent. Particularly useful agents include, e.g., anti-inflammatory agents, antimicrobial agents, antifungal agents, antiviral agents, antiproliferative agents, analgesics, anesthetics, immunomodulators, or lubricants.

If more than one agent is employed (e.g., a tribonectin and a biologically active agent), the agents may be applied to the surface of the biocompatible device separately or may be admixed and applied together. The agents described herein may be admixed with additional active or inert ingredients, e.g., in conventional polymeric carriers for the coating of the biocompatible device. A polymeric carrier may be any compatible, non-toxic substance suitable for the administration of the agents to the surface of the device.

As described herein, the surface layer of the biocompatible device may further include an additional biologically active agent. This agent may be, e.g., an anti-inflammatory agent, antimicrobial agent, antifungal agent, antiviral agent, antiproliferative agent, analgesic, anesthetic, immunomodulator, or a lubricant.

Anti-Inflammatory Agents

Any suitable anti-inflammatory agent may be included in the surface layer of the biocompatible device. Suitable anti-inflammatory agents include, e.g., non-steroidal anti-inflammatory drugs (e.g., ibuprofen or tacrolimus), cyclooxygenase-2-specific inhibitors such as rofecoxib (Vioxx®) and celecoxib (Celebrex®), topical glucocorticoid agents, and specific cytokines directed at T lymphocyte function. Additional suitable anti-inflammatory agents include flubiprofen, diclofenac, and ketarolac. Exemplary anti-inflammatory agents may be found in, e.g., U.S. Pat. Nos. 7,112,578 and 7,199,119.

Antimicrobial Agents

Any of the many known antimicrobial agents may be included in the surface later of the biocompatible device. Antimicrobial agents include antibacterials, antifungals, and antivirals.

Examples of antibacterial agents (antibiotics) include, e.g., penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, minocycline, and tetracycline), aminoglycosides (e.g., amikacin, gentamycin, kanamycin, neomycin, streptomycin, and tobramycin), macrolides (e.g., azithromycin, clarithromycin, and erythromycin), fluoroquinolones (e.g., ciprofloxacin, lomefloxacin, moxifloxacin, and norfloxacin), and other antibiotics including chloramphenicol, clindamycin, cycloserine, isoniazid, rifampin, and vancomycin. Exemplary antimicrobial agents may be found in, e.g., U.S. Pat. Nos. 6,830,745 and 7,056,917.

Examples of antiviral agents include, e.g., 1-β-D-ribofuranosyl-1,2,4-triazole-3 carboxamide (ribavirin), 9-2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir. Exemplary antiviral agents may be found in, e.g., U.S. Pat. Nos. 6,093,550 and 6,894,033.

Antifungal agents include both fungicidal and fungistatic agents, e.g., amphotericin B, butylparaben, clindamycin, econaxole, fluconazole, flucytosine, griseofulvin, nystatin, and ketoconazole. Exemplary antifungal agents may be found in, e.g., U.S. Pat. Nos. 5,627,153 and 7,125,842.

Antiproliferative Agents

Exemplary antiproliferative agents which may be used in the devices and methods of the invention include, e.g., mechlorethamine, cyclophosphamide, ifosfamide, melphalan, chlorambucil, uracil mustard, estramustine, mitomycin C, AZQ, thiotepa, busulfan, hepsulfam, carmustine, lomustine, semustine, streptozocin, dacarbazine, cisplatin, carboplatin, procarbazine, methotrexate, trimetrexate, fluouracil, floxuridine, cytarabine, fludarabine, capecitabine, azacitidine, thioguanine, mercaptopurine, allopurine, cladribine, gemcitabine, pentostatin, vinblastine, vincristine, etoposide, teniposide, topotecan, irinotecan, camptothecin, 9-aminocamptothecin, paclitaxel, docetaxel, daunorubicin, doxorubicin, dactinomycin, idarubincin, plicamycin, mitomycin, amsacrine, bleomycin, aminoglutethimide, anastrozole, finasteride, ketoconazole, tamoxifen, flutamide, leuprolide, goserelin, and Gleevec™ (Novartis).

Analgesics and Anesthetics

Any of the commonly used analgesics and anesthetics may be used in the invention. Examples of useful anesthetics include, e.g., procaine, lidocaine, tetracaine, dibucaine, benzocaine, p-buthylaminobenzoic acid 2-(diethylamino) ethyl ester HCl, mepivacaine, piperocaine, and dyclonine. Exemplary anesthetics may be found in, e.g., U.S. Pat. Nos. 6,562,363 and 6,569,839.

Analgesics include opioids such as morphine, codeine, hydrocodone, and oxycodone. Any of these analgesics may also be co-formulated with other compounds having analgesic or anti-inflammatory properties, such as acetaminophen, aspirin, codeine, naproxen, and ibuprofen. Exemplary analgesics may be found in, e.g., U.S. Pat. Nos. 6,869,974 and 7,202,259.

Immunomodulatory Agents

Examples of useful immunomodulatory agents include, e.g., non-steroidal immunophilin-dependent immunosuppressants, e.g., ascomycin, cyclosporine (e.g., Restasis), everolimus, pimecrolimus, rapamycin, and tacrolimus. Also included are steroids, e.g., beclomethasone, budesonide, dexamethasone, fluorometholone, fluticasone, hydrocortisone, loteprednol etabonate, medrysone, rimexolone, and triamcinolone. Exemplary steroids may be found in, e.g., U.S. Pat. Nos. 5,837,698 and 6,909,007.

Lubricants

Examples of lubricants useful as additional biologically active agents include hyaluronic acid, sodium hyaluronate, proteoglycans, chondroitin sulfate, cellulose derivatives, hydroxypropylmethyl cellulose, carboxymethyl cellulose, methyl cellulose, hydroxyethyl cellulose, collagen, viscosifiers, polyvinyl alcohol, polyvinylpyrrolidone, and carboxyvinyl polymers. Exemplary lubricants may be found in, e.g., U.S. Pat. No. 7,037,469.

Hyaluronic acid may be used as a lubricant on the surface coating of the biocompatible device of the invention described herein. Hyaluronic acid is a naturally-occurring, cross-linked polysaccharide containing alternating N-acetyl-D-glucosamine and D-glucuronic acid monosaccharide units. In the invention described herein, hyaluronic acid may be present in the coating of the biocompatible device at a concentration of between 0.1 mg/ml and 50.0 mg/ml. The hyaluronic acid used in the inventioned described herein may be, e.g., isolated from a natural source, produced in vitro, or may be chemically synthesized (see, e.g., U.S. Pat. Nos. 5,563,051, 6,489,467, 6,537,795, and 7,105,320, hereby incorporated by reference).

### EXAMPLE

The present invention is illustrated by the following example, which is in no way intended to be limiting of the invention.

### Example 1

### Preparation of a Tribonectin-Coated Biocompatible Device

The surface of two triluminal central venous catheters were contacted with a lubricin solution at a concentration of 200 μg/ml in normal saline. The lubricin-coated catheters were then incubated with a standardized bacterial solution for three hours with gentle agitation at room temperature. Two control triluminal central venous catheters were contacted with physiologic saline without lubricin and were then incubated with a standardized bacterial solution for three hours with gentle agitation at room temperature. After incubation with the bacterial solution, both the lubricin-coated catheters and the control catheters were triple-washed to remove non-attached bacteria. The catheters were then divided into four segments per catheter. These segments were labeled A (control) and B (lubricin-coated), as shown in Table 1. The catheter segments were cultured according to standard microbiological protocols in a blinded study. Colony-forming unit counts per milliliter (CFU/ml) were determined at 24, 48, and 72 hours post inoculation. The results are shown in Table 1.

TABLE 1

| | After | | |
|---|---|---|---|
| | 24 hours | After 48 hours | After 72 hours |
| Specimen | (CFU/ml) | (CFU/ml) | (CFU/ml) |
| A1 | 1-10 | >100 | >100 |
| A2 | 50-100 | >100 | >100 |
| A3 | 1-10 | >100 | >100 |
| A4 | 50-100 | >100 | >100 |
| A5 | 1-10 | >100 | >100 |
| A6 | 1-10 | >100 | >100 |
| A7 | 1-10 | 100 | >100 |
| A8 | 1-10 | 100 | >100 |
| B1 | <10 (6) | 10-50 | 10-50 |
| B2 | 1-10 | 50-100 | 50-100 |
| B3 | <10 (1) | 10-50 | 50-100 |
| B4 | 10-50 | >100 | >100 |
| B5 | 10-50 | >100 | >100 |
| B6 | 10-50 | 50-100 | 50-100 |
| B7 | 10-50 | 50-100 | 50-100 |
| B8 | 10-50 | 50-100 | 50-100 |

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1. A non-diarthrodial, biocompatible device adapted for use within the body of a mammal, said device comprising a surface layer coating comprising a tribonectin.

2. The device of claim 1, wherein said device is used for the reduction of microbial growth on the surface of said device for use within said mammal in need thereof.

3. The device of claim 1, wherein said coating comprises a biologically active agent.

4. The device of claim 1, wherein said device is sterile.

5. The device of claim 1, wherein said tribonectin is present in an amount sufficient to reduce microbial growth on the surface of said device when said device is used within said mammal.

6. The device of claim 1, wherein said coating comprises said tribonectin at a concentration of between 0.1 μg/ml to 1.0 mg/ml.

7. The device of claim 6, wherein said coating comprises said tribonectin at a concentration of 0.2 mg/ml.

8. The device of claim 1, wherein said tribonectin is lubricin or a biologically active fragment thereof.

9. The device of claim 3, wherein said biologically active agent is an anti-inflammatory agent, antimicrobial agent, antifungal agent, antiviral agent, antiproliferative agent, analgesic, anesthetic, immunomodulator, or a lubricant.

10. The device of claim 9, wherein said anti-inflammatory agent is ibuprofen, tacrolimus, rofecoxib, celecoxib, flubiprofen, diclofenac, or ketarolac.

11. The device of claim 9, wherein said antimicrobial agent is penicillin, ampicillin, methicillin, oxacillin, amoxicillin, cefadroxil, ceforanid, cefotaxime, ceftriaxone, doxycycline, minocycline, tetracycline, amikacin, gentamycin, kanamycin, neomycin, streptomycin, tobramycin, azithromycin, clarithromycin, erythromycin, ciprofloxacin, lomefloxacin, moxifloxacin, norfloxacin, chloramphenicol, clindamycin, cycloserine, isoniazid, rifampin, or vancomycin.

12. The device of claim 9, wherein said antiviral agent is ribavirin, 9-2-hydroxy-ethoxy methylguanine, adamantanamine, 5-iodo-2′-deoxyuridine, trifluorothymidine, interferon, adenine arabinoside, acyclovir, penciclovir, valacyclovir, or ganciclovir.

13. The device of claim 9, wherein said antiproliferative agent is asparaginase, bleomycin, busulfan carmustine (BCNU), chlorambucil, cladribine (2-CdA), CPT11, cyclophosphamide, cytarabine (Ara-C), dacarbazine, daunorubicin, dexamethasone, doxorubicin (adriamycin), etoposide, fludarabine, 5-fluorouracil (5FU), hydroxyurea, idarubicin, ifosfamide, interferon-α (native or recombinant), levamisole, lomustine (CCNU), mechlorethamine (nitrogen mustard), melphalan, mercaptopurine, methotrexate, mitomycin, mitoxantrone, paclitaxel, pentostatin, prednisone, procarbazine, tamoxifen, taxol-related compounds, 6-thioguanine, topotecan, vinblastine, or vincristine.

14. The device of claim 9, wherein said antifungal agent is amphotericin B, butylparaben, clindamycin, econaxole, fluconazole, flucytosine, griseofulvin, nystatin, or ketoconazole.

15. The device of claim 9, wherein said analgesic is morphine, codeine, hydrocodone, oxycodone, acetaminophen, aspirin, codeine, naproxen, or ibuprofen.

16. The device of claim 9, wherein said anesthetic is procaine, lidocaine, tetracaine, dibucaine, benzocaine, p-buthylaminobenzoic acid 2-(diethylamino) ethyl ester HCl, mepivacaine, piperocaine, or dyclonine.

17. The device of claim 9, wherein said lubricant is hyaluronic acid, a proteoglycan, chondroitin sulfate, a cellulose derivative, hydroxypropylmethyl cellulose, carboxymethyl cellulose, methyl cellulose, hydroxyethyl cellulose, collagen, a viscosifier, polyvinyl alcohol, polyvinylpyrrolidone, or a carboxyvinyl polymer.

18. The device of claim 9, wherein said lubricant is hyaluronic acid.

19. The device of claim 18, wherein said hyaluronic acid is present in said coating at a concentration of between 0.1 mg/ml to 50.0 mg/ml.

20. The device of claim 9, wherein said immunomodulator is ascomycin, cyclosporine, everolimus, pimecrolimus, rapamycin, tacrolimus, beclomethasone, budesonide, dexamethasone, fluorometholone, fluticasone, hydrocortisone, loteprednol etabonate, medrysone, rimexolone, or triamcinolone.

21. The device of claim 1, wherein said device is a catheter, stent, intraocular lens, dialysis graft, pacemaker lead, implantable defibrillator, suture, suture anchor, staple, clamp, screw, plate, shunt, bone pin, vertebral disk, hemostatic barrier, tissue adhesive or sealant, tissue scaffold, bone substitute, anastomosis device, intraluminal device, angioplasty device, drug-delivery device, non-diarthrodial prosthetic implant, vascular implant, or vascular support.

22. A method of making a non-diarthrodial, biocompatible device adapted for use within the body of a mammal comprising the steps of coating a surface layer of said device with a composition comprising a tribonectin.

23. The method of claim 22, where said composition reduces microbial growth on the surface of said device.

24. The method of claim 22, wherein said composition further comprises a biologically active agent.

25. The method of claim 22, wherein said device is sterile.

26. The method of claim 22, wherein said tribonectin is present in an amount sufficient to reduce microbial growth on the surface of said device when said device is used within said mammal.

27. The method of claim 22, wherein said composition comprises said tribonectin at a concentration of between 0.1 μg/ml to 1.0 mg/ml.

28. The method of claim 22, wherein said tribonectin is lubricin biologically active fragments thereof.

29. The method of claim 24, wherein said biologically active agent is an anti-inflammatory agent, antimicrobial agent, antifungal agent, antiviral agent, antiproliferative agent, analgesic, anesthetic, immunomodulator, or a lubricant.

30. The method of claim 29, wherein said anti-inflammatory agent is ibuprofen, tacrolimus, rofecoxib, celecoxib, flubiprofen, diclofenac, or ketarolac.

31. The method of claim 29, wherein said antimicrobial agent is penicillin, ampicillin, methicillin, oxacillin, amoxicillin, cefadroxil, ceforanid, cefotaxime, ceftriaxone, doxycycline, minocycline, tetracycline, amikacin, gentamycin, kanamycin, neomycin, streptomycin, tobramycin, azithromycin, clarithromycin, erythromycin, ciprofloxacin, lomefloxacin, moxifloxacin, norfloxacin, chloramphenicol, clindamycin, cycloserine, isoniazid, rifampin, or vancomycin.

32. The method of claim 29, wherein said antiviral agent is ribavirin, 9-2-hydroxy-ethoxy methylguanine, adamantanamine, 5-iodo-2′-deoxyuridine, trifluorothymidine, interferon, adenine arabinoside, acyclovir, penciclovir, valacyclovir, or ganciclovir.

33. The method of claim 29, wherein said antiproliferative agent is asparaginase, bleomycin, busulfan carmustine (BCNU), chlorambucil, cladribine (2-CdA), CPT11 cyclophosphamide, cytarabine (Ara-C), dacarbazine, daunorubicin, dexamethasone, doxorubicin (adriamycin), etoposide, fludarabine, 5-fluorouracil (5FU), hydroxyurea, idarubicin, ifosfamide, interferon-α (native or recombinant), levamisole, lomustine (CCNU), mechlorethamine (nitrogen mustard), melphalan, mercaptopurine, methotrexate, mitomycin, mitoxantrone, paclitaxel, pentostatin, prednisone, procarbazine, tamoxifen, taxol-related compounds, 6-thioguanine, topotecan, vinblastine, or vincristine.

34. The method of claim 29, wherein said antifungal agent is amphotericin B, butylparaben, clindamycin, econaxole, fluconazole, flucytosine, griseofulvin, nystatin, or ketoconazole.

35. The method of claim 29, wherein said analgesic is morphine, codeine, hydrocodone, oxycodone, acetaminophen, aspirin, codeine, naproxen, or ibuprofen.

36. The method of claim 29, wherein said anesthetic is procaine, lidocaine, tetracaine, dibucaine, benzocaine, p-buthylaminobenzoic acid 2-(diethylamino) ethyl ester HCl, mepivacaine, piperocaine, or dyclonine.

37. The method of claim 29, wherein said lubricant is hyaluronic acid, a proteoglycan, chondroitin sulfate, a cellulose derivative, hydroxypropylmethyl cellulose, carboxymethyl cellulose, methyl cellulose, hydroxyethyl cellulose, collagen, a viscosifier, polyvinyl alcohol, polyvinylpyrrolidone, or a carboxyvinyl polymer.

38. The method of claim 29, wherein said lubricant is hyaluronic acid.

39. The method of claim 38, wherein said hyaluronic acid is present in said coating at a concentration of between 0.1 mg/ml to 50.0 mg/ml.

40. The method of claim 29, wherein said immunomodulator is ascomycin, cyclosporine, everolimus, pimecrolimus, rapamycin, tacrolimus, beclomethasone, budesonide, dexamethasone, fluorometholone, fluticasone, hydrocortisone, loteprednol etabonate, medrysone, rimexolone, or triamcinolone.

41. The method of claim 22, wherein said device is a catheter, stent, intraocular lens, dialysis graft, pacemaker lead, implantable defibrillator, suture, suture anchor, staple, clamp, screw, plate, shunt, bone pin, vertebral disk, hemostatic barrier, tissue adhesive or sealant, tissue scaffold, bone substitute, anastomosis device, intraluminal device, angioplasty device, drug-delivery device, non-arthrodial prosthetic implant, vascular implant, or vascular support.


SEQUENCE LISTING (3 sequences)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIWKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTAPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTSDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGEMTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - DNA, 5041 bp, Homo sapiens
gcggccgcgactattcggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaatttggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccgctcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaatgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaaaaaaaaaaaaaaaa

SEQ ID NO: 3 - protein, 7 aa, Artificial Sequence
  synthetic
KEPAPTT